SEQUENCE LISTING (12 sequences)

SEQ ID NO: 1 - DNA, 21 bp, Artificial Sequence
  Description of Artificial Sequence Synthetic primer
tctcccagctgttcttcatgc

SEQ ID NO: 2 - DNA, 20 bp, Artificial Sequence
  Description of Artificial Sequence Synthetic primer
ccagttgaaggtactccccg

SEQ ID NO: 3 - DNA, 20 bp, Artificial Sequence
  Description of Artificial Sequence Synthetic primer
ggcctccaaggagtaagacc

SEQ ID NO: 4 - DNA, 20 bp, Artificial Sequence
  Description of Artificial Sequence Synthetic primer
aggggtctacatggcaactg

SEQ ID NO: 5 - DNA, 32 bp, Artificial Sequence
  Description of Artificial Sequence Synthetic primer
atctggcaccacaccttctacaatgagctgcg

SEQ ID NO: 6 - DNA, 32 bp, Artificial Sequence
  Description of Artificial Sequence Synthetic primer
cgtcatactcctgcttgctgatccacatctgc

SEQ ID NO: 7 - RNA, 19 nt, Artificial Sequence
  Description of Artificial Sequence Synthetic oligonucleotide
gccgucucaagaagugcuu

SEQ ID NO: 8 - RNA, 19 nt, Artificial Sequence
  Description of Artificial Sequence Synthetic oligonucleotide
cauugagacacugaucaga

SEQ ID NO: 9 - RNA, 19 nt, Artificial Sequence
  Description of Artificial Sequence Synthetic oligonucleotide
gcaagcauuacggugucuu

SEQ ID NO: 10 - RNA, 19 nt, Artificial Sequence
  Description of Artificial Sequence Synthetic oligonucleotide
ccccuagcaugaacuugug

SEQ ID NO: 11 - DNA, 64 bp, Artificial Sequence
  Description of Artificial Sequence Synthetic oligonucleotide
gatccgcattacggtgtcttcaccttcaagagaggtgaagacaccgtaatgcttttttctagag

SEQ ID NO: 12 - DNA, 65 bp, Artificial Sequence
  Description of Artificial Sequence Synthetic oligonucleotide
gatccgcctctggacacgtaacctattcaagagataggttacgtgtccagaggttttttctagag

(19) United States
(12) Patent Application Publication
(10) Pub. No.: US 2021/0317180 A1
(43) Pub. Date: Oct. 14, 2021

(54) NR2F6 INHIBITED CHIMERIC ANTIGEN RECEPTOR CELLS

(71) Applicant: Regen Biopharma, Inc., La Mesa, CA (US)

(72) Inventors: Thomas Ichim, San Diego, CA (US); David Koos, La Mesa, CA (US)

(21) Appl. No.: 15/351,414

(22) Filed: Nov. 14, 2016

Related U.S. Application Data
(60) Provisional application No. 62/254,330, filed on Nov. 12, 2015.

(51) Int. Cl. C07K 14/725; A61K 35/17; C12N 15/113; C07K 16/30

(52) U.S. Cl. CPC C07K 14/7051; A61K 35/17; C12N 15/113; C07K 16/30; C12N 2320/30; C12N 2310/14; C07K 2317/622; C07K 2317/55; C07K 2319/02; C07K 2319/03; C12N 2310/531

(57) ABSTRACT

Disclosed are compositions of matter, cells, and methodologies for generation of chimeric antigen receptor (CAR) cells with inhibited or absent NR2F6 activity. In one embodiment, a CAR possessing affinity to a tumor antigen is transfected onto T cells that possess reduced or absent NR2F6 activity, said reduction or absence of NR2F6 activity leading to increased production of cytokines associated with inhibition of tumor growth, metastasis or angiogenesis, and/or augmentation of tumor cytotoxicity. Inhibition of NR2F6 activity may be performed ex vivo on said T cells or in vivo by administration of small molecule inhibitors, siRNA, shRNA or gene editing. In some embodiments other immune cells are substituted for CAR-T cells.

### CROSS-REFERENCE TO RELATED APPLICATIONS

The present invention claims priority to provisional U.S. patent application No. 62/254,330, filed Nov. 12, 2015, which is hereby incorporated in its entirety including all tables, figures, and claims.

### BACKGROUND OF THE INVENTION

The standard of treatments for cancer are surgery, radiation therapy, and chemotherapy. Unfortunately, these approaches are often not curative and are associated with extremely high toxicity and adverse effects. Immunotherapy which uses the body's immune system, either directly or indirectly, to shrink or eradicate cancer has been studied for many years as an adjunct to conventional cancer therapy. It is believed that the human immune system is an untapped resource for cancer therapy and that effective treatment can be developed once the components of the immune system are properly harnessed. As key immunoregulatory molecules and signals of immunity are identified and prepared as therapeutic reagents, the clinical effectiveness of such reagents can be tested using established cancer models. Immunotherapeutic strategies include administration of vaccines, activated cells, antibodies, cytokines, chemokines, as well as small molecular inhibitors, anti-sense oligonucleotides, and gene therapy. It is believed by many that immunotherapy offers the potential for treatment of cancer without the toxicities associated with current approaches to cancer therapy.

Unfortunately while numerous studies have demonstrated that immune cells are capable of killing cancers in vitro or at a small scale in vivo, the power of immunotherapy has not been fully utilized due to: a) lack of ability to expand immunological cells capable of specifically killing tumors; and b) tumor initiated defense mechanisms.

Chimeric antigen receptor (CAR) T cells overcome some of these limitations. CAR T cells do not need MHC I presentation of antigen since they usually have an antibody domain connected to T cell receptor (TCR) signaling molecules. Accordingly, CAR T cells are not limited by need for MHC antigen presentation. This is important since many tumors downregulate MHC or associated antigen processing machinery such as TAP.

The current use of CAR-T cells has limitations in solid tumors. In part this is due to inhibition of immune molecules by tumor derived immune suppressive factors. The current invention seeks to potentiate CAR-T and CAR-NK cells through silencing or substantially inhibiting activity of the T cell and NK cell inhibitor NR2F6.

### FIELD OF THE INVENTION

The invention pertains to the field of cancer immunotherapy, more specifically, the invention pertains to the use of CAR T cells and CAR NK cells in treatment of cancer, more specifically, the invention pertains to the area of genetically modified CAR T cells.

### DESCRIPTION OF THE INVENTION

The invention provides means of augmenting efficacy of CAR-T cells and CAR-NK cells through silencing or substantially inhibiting NR2F6 activity. Said inhibition may be performed in vitro, ex vivo, or in vivo. Means of inhibition include specific siRNA, gene editing and shRNA, which in some embodiments are preferential for ex vivo inhibition of NR2F6, or in vivo inhibition through use of small molecules.

For the purpose of defining the invention, terms are presented below. Unless defined differently, all technical and scientific terms used herein have the same meanings as commonly understood by one of skill in the art to which the disclosed invention belongs. In particular, the following terms and phrases have the following meaning.

“Treating a cancer”, “inhibiting cancer”, “reducing cancer growth” refers to inhibiting or preventing oncogenic activity of cancer cells. Oncogenic activity can comprise inhibiting migration, invasion, drug resistance, cell survival, anchorage-independent growth, non-responsiveness to cell death signals, angiogenesis, or combinations thereof of the cancer cells. The terms “cancer”, “cancer cell”, “tumor”, and “tumor cell” are used interchangeably herein and refer generally to a group of diseases characterized by uncontrolled, abnormal growth of cells (e.g., a neoplasia). In some forms of cancer, the cancer cells can spread locally or through the bloodstream and lymphatic system to other parts of the body (“metastatic cancer”). “Ex vivo activated lymphocytes”, “lymphocytes with enhanced antitumor activity” and “dendritic cell cytokine induced killers” are terms used interchangeably to refer to composition of cells that have been activated ex vivo and subsequently reintroduced within the context of the current invention. Although the word “lymphocyte” is used, this also includes heterogenous cells that have been expanded during the ex vivo culturing process including dendritic cells, NKT cells, gamma delta T cells, and various other innate and adaptive immune cells. As used herein, “cancer” refers to all types of cancer or neoplasm or malignant tumors found in animals, including leukemias, carcinomas and sarcomas. Examples of cancers are cancer of the brain, melanoma, bladder, breast, cervix, colon, head and neck, kidney, lung, non-small cell lung, mesothelioma, ovary, prostate, sarcoma, stomach, uterus and Medulloblastoma. The term “leukemia” is meant broadly progressive, malignant diseases of the hematopoietic organs/systems and is generally characterized by a distorted proliferation and development of leukocytes and their precursors in the blood and bone marrow. Leukemia diseases include, for example, acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, basophilic leukemia, blast cell leukemia, bovine leukemia, chronic myelocytic leukemia, leukemia cutis, embryonal leukemia, eosinophilic leukemia, Gross' leukemia, Rieder cell leukemia, Schilling's leukemia, stem cell leukemia, subleukemic leukemia, undifferentiated cell leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, and promyelocytic leukemi.

The term “carcinoma” refers to a malignant new growth made up of epithelial cells tending to infiltrate the surrounding tissues, and/or resist physiological and non-physiological cell death signals and give rise to metastases. Exemplary carcinomas include, for example, acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiennoid carcinoma, carcinoma epitheliale adenoides, exophytic carcinoma, carcinoma ex ulcere, carcinoma fibrosum, gelatiniform carcinoma, gelatinous carcinoma, giant cell carcinoma, signet-ring cell carcinoma, carcinoma simplex, small-cell carcinoma, solanoid carcinoma, spheroidal cell carcinoma, spindle cell carcinoma, carcinoma spongiosum, squamous carcinoma, squamous cell carcinoma, string carcinoma, carcinoma telangiectaticum, carcinoma telangiectodes, transitional cell carcinoma, carcinoma tuberosum, tuberous carcinoma, verrmcous carcinoma, carcinoma villosum, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypemephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, carcinoma molle, mucinous carcinoma, carcinoma muciparum, carcinoma mucocellulare, mucoepidermoid carcinoma, carcinoma mucosum, mucous carcinoma, carcinoma myxomatodes, naspharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, schneiderian carcinoma, scirrhous carcinoma, and carcinoma scroti, The term “sarcoma” generally refers to a tumor which is made up of a substance like the embryonic connective tissue and is generally composed of closely packed cells embedded in a fibrillar, heterogeneous, or homogeneous substance. Sarcomas include, chondrosarcoma, fibrosarcoma, lymphosarcoma, melanosarcoma, myxosarcoma, osteosarcoma, endometrial sarcoma, stromal sarcoma, Ewing's sarcoma, fascial sarcoma, fibroblastic sarcoma, giant cell sarcoma, Abemethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilns' tumor sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma. Additional exemplary neoplasias include, for example, Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic insulanoma, malignant carcinoid, premalignant skin lesions, testicular cancer, lymphomas, thyroid cancer, neuroblastoma, esophageal cancer, genitourinary tract cancer, malignant hypercalcemia, cervical cancer, endometrial cancer, and adrenal cortical cancer.

In some particular embodiments of the invention, the cancer treated is a melanoma. The term “melanoma” is taken to mean a tumor arising from the melanocytic system of the skin and other organs. Melanomas include, for example, Harding-Passey melanoma, juvenile melanoma, lentigo maligna melanoma, malignant melanoma, acral-lentiginous melanoma, amelanotic melanoma, benign juvenile melanoma, Cloudman's melanoma, S91 melanoma, nodular melanoma subungal melanoma, and superficial spreading melanoma. The term “polypeptide” is used interchangeably with “peptide”, “altered peptide ligand”, and “flourocarbonated peptides.” The term “pharmaceutically acceptable carrier” includes any and all solvents, dispersion media, coatings, antibacterial and antifungal agents, isotonic and absorption delaying agents, and the like. The use of such media and agents for pharmaceutically active substances is well known in the art. Except insofar as any conventional media or agent is incompatible with the active compound, use thereof in the therapeutic compositions is contemplated. Supplementary active compounds can also be incorporated into the compositions.

The term “T cell” is also referred to as T lymphocyte, and means a cell derived from thymus among lymphocytes involved in an immune response. The T cell includes any of a CD8-positive T cell (cytotoxic T cell: CTL), a CD4-positive T cell (helper T cell), a suppressor T cell, a regulatory T cell such as a controlling T cell, an effector cell, a naive T cell, a memory T cell, an .alpha..beta.T cell expressing TCR .alpha. and .beta. chains, and a .gamma..delta.T cell expressing TCR .gamma. and .delta. chains. The T cell includes a precursor cell of a T cell in which differentiation into a T cell is directed. Examples of “cell populations containing T cells” include, in addition to body fluids such as blood (peripheral blood, umbilical blood etc.) and bone marrow fluids, cell populations containing peripheral blood mononuclear cells (PBMC), hematopoietic cells, hematopoietic stem cells, umbilical blood mononuclear cells etc., which have been collected, isolated, purified or induced from the body fluids. Further, a variety of cell populations containing T cells and derived from hematopoietic cells can be used in the present invention. These cells may have been activated by cytokine such as IL-2 in vivo or ex vivo. As these cells, any of cells collected from a living body, or cells obtained via ex vivo culture, for example, a T cell population obtained by the method of the present invention as it is, or obtained by freeze preservation, can be used. The term “antibody” is meant to include both intact molecules as well as fragments thereof that include the antigen-binding site. Whole antibody structure is often given as H.sub.2L.sub.2 and refers to the fact that antibodies commonly comprise 2 light (L) amino acid chains and 2 heavy (H) amino acid chains. Both chains have regions capable of interacting with a structurally complementary antigenic target. The regions interacting with the target are referred to as “variable” or “V” regions and are characterized by differences in amino acid sequence from antibodies of different antigenic specificity. The variable regions of either H or L chains contains the amino acid sequences capable of specifically binding to antigenic targets. Within these sequences are smaller sequences dubbed “hypervariable” because of their extreme variability between antibodies of differing specificity. Such hypervariable regions are also referred to as “complementarity determining regions” or “CDR” regions. These CDR regions account for the basic specificity of the antibody for a particular antigenic determinant structure. The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all antibodies each have 3 CDR regions, each non-contiguous with the others (termed L1, L2, L3, H1, H2, H3) for the respective light (L) and heavy (H) chains. The antibodies disclosed according to the invention may also be wholly synthetic, wherein the polypeptide chains of the antibodies are synthesized and, possibly, optimized for binding to the polypeptides disclosed herein as being receptors. Such antibodies may be chimeric or humanized antibodies and may be fully tetrameric in structure, or may be dimeric and comprise only a single heavy and a single light chain.

The term “effective amount” or “therapeutically effective amount” means a dosage sufficient to treat, inhibit, or alleviate one or more symptoms of a disease state being treated or to otherwise provide a desired pharmacologic and/or physiologic effect, especially enhancing T cell response to a selected antigen. The precise dosage will vary according to a variety of factors such as subject-dependent variables (e.g., age, immune system health, etc.), the disease, and the treatment being administered.

The terms “individual”, “host”, “subject”, and “patient” are used interchangeably herein, and refer to a mammal, including, but not limited to, primates, for example, human beings, as well as rodents, such as mice and rats, and other laboratory animals.

As used herein, the term “treatment regimen” refers to a treatment of a disease or a method for achieving a desired physiological change, such as increased or decreased response of the immune system to an antigen or immunogen, such as an increase or decrease in the number or activity of one or more cells, or cell types, that are involved in such response, wherein said treatment or method comprises administering to an animal, such as a mammal, especially a human being, a sufficient amount of two or more chemical agents or components of said regimen to effectively treat a disease or to produce said physiological change, wherein said chemical agents or components are administered together, such as part of the same composition, or administered separately and independently at the same time or at different times (i.e., administration of each agent or component is separated by a finite period of time from one or more of the agents or components) and where administration of said one or more agents or components achieves a result greater than that of any of said agents or components when administered alone or in isolation.

The term “anergy” and “unresponsiveness” includes unresponsiveness to an immune cell to stimulation, for example, stimulation by an activation receptor or cytokine. The anergy may occur due to, for example, exposure to an immune suppressor or exposure to an antigen in a high dose. Such anergy is generally antigen-specific, and continues even after completion of exposure to a tolerized antigen. For example, the anergy in a T cell and/or NK cell is characterized by failure of production of cytokine, for example, interleukin (IL)-2. The T cell anergy and/or NK cell anergy occurs in part when a first signal (signal via TCR or CD-3) is received in the absence of a second signal (costimulatory signal) upon exposure of a T cell and/or NK cell to an antigen. The term “enhanced function of a T cell”, “enhanced cytotoxicity” and “augmented activity” means that the effector function of the T cell and/or NK cell is improved. The enhanced function of the T cell and/or NK cell, which does not limit the present invention, includes an improvement in the proliferation rate of the T cell and/or NK cell, an increase in the production amount of cytokine, or an improvement in cytotoxity. Further, the enhanced function of the T cell and/or NK cell includes cancellation and suppression of tolerance of the T cell and/or NK cell in the suppressed state such as the anergy (unresponsive) state, or the rest state, that is, transfer of the T cell and/or NK cell from the suppressed state into the state where the T cell and/or NK cell responds to stimulation from the outside. The term “expression” means generation of mRNA by transcription from nucleic acids such as genes, polynucleotides, and oligonucleotides, or generation of a protein or a polypeptide by transcription from mRNA. Expression may be detected by means including RT-PCR, Northern Blot, or in situ hybridization, “Suppression of expression” refers to a decrease of a transcription product or a translation product in a significant amount as compared with the case of no suppression. The suppression of expression herein shows, for example, a decrease of a transcription product or a translation product in an amount of 30% or more, preferably 50% or more, more preferably 70% or more, and further preferably 90% or more.

In one embodiment the invention provides a CAR-T or CAR-NK cell comprising an extracellular and intracellular domain, wherein said CAR cell possesses sufficiently inhibited NR2F6 activity in order to allow for enhanced costimulation as compared to a CAR cell that possesses non-altered NR2F6 activity. The extracellular domain comprises a target-specific binding element otherwise referred to as an antigen binding domain. In some embodiments, the extracellular domain also comprises a hinge domain. The intracellular domain or otherwise the cytoplasmic domain comprises, a costimulatory signaling region and a zeta chain portion. The costimulatory signaling region refers to a portion of the CAR comprising the intracellular domain of a costimulatory molecule. Costimulatory molecules are cell surface molecules other than antigens receptors or their ligands that are required for an efficient response of lymphocytes to antigen. Between the extracellular domain and the transmembrane domain of the CAR, or between the cytoplasmic domain and the transmembrane domain of the CAR, there may be incorporated a spacer domain. As used herein, the term “spacer domain” generally means any oligo- or polypeptide that functions to link the transmembrane domain to, either the extracellular domain or, the cytoplasmic domain in the polypeptide chain. A spacer domain may comprise up to 300 amino acids, preferably 10 to 100 amino acids and most preferably 25 to 50 amino acids. The present invention includes retroviral and lentiviral vector constructs expressing a CAR that can be directly transduced into a cell. The present invention also includes an RNA construct that can be directly transfected into a cell. The NR2F6 modulation serves, in one embodiment of the invention, as a means of enhancing costimulatory signals. A method for generating mRNA for use in transfection involves in vitro transcription (IVT) of a template with specially designed primers, followed by polyA addition, to produce a construct containing 3′ and 5′ untranslated sequence (“UTR”), a 5′ cap and/or Internal Ribosome Entry Site (IRES), the gene to be expressed, and a polyA tail, typically 50-2000 bases in length. RNA so produced can efficiently transfect different kinds of cells. In one embodiment, the template includes sequences for the CAR. Preferably, the CAR comprises an extracellular domain, a transmembrane domain and a cytoplasmic domain. The extracellular domain and transmembrane domain can be derived from any desired source of such domains. The extracellular domain may be obtained from any of the wide variety of extracellular domains or secreted proteins associated with ligand binding and/or signal transduction. In one embodiment, the extracellular domain may consist of an Ig heavy chain which may in turn be covalently associated with Ig light chain by virtue of the presence of CH1 and hinge regions, or may become covalently associated with other Ig heavy/light chain complexes by virtue of the presence of hinge, CH2 and CH3 domains. In the latter case, the heavy/light chain complex that becomes joined to the chimeric construct may constitute an antibody with a specificity distinct from the antibody specificity of the chimeric construct. Depending on the function of the antibody, the desired structure and the signal transduction, the entire chain may be used or a truncated chain may be used, where all or a part of the CH1, CH2, or CH3 domains may be removed or all or part of the hinge region may be removed.

The present invention comprises an antigen binding domain that binds to a stromal cell antigen. As discussed elsewhere herein, the present invention provides that targeting of the stromal cells existing in the in the tumor microenvironment allows for the reduction and/or elimination of the tumor. In one embodiment, the antigen binding domain comprises a domain directed to a tumor antigen. Said tumor antigen is expressed on a vast majority of stromal cells in many types of human carcinomas. In one embodiment, the CAR may be one for which a specific monoclonal antibody currently exists or can be generated in the future. The tumor may be of any type, wherein the tumor microenvironment includes stromal cells. In one embodiment, the tumor is a carcinoma. In one embodiment, the retroviral or lentiviral vector comprises a CAR designed to be directed to a tumor antigen by way of engineering an anti-antigen domain into the CAR. In another embodiment, the template for the RNA CAR is designed to be directed to a tumor antigen by way of engineering an anti-tumor antigen domain into the CAR. The CAR of the invention can be engineered to include any anti-tumor antigen moiety that is specific to said tumor antigen. The antigen binding domain can be any domain that binds to the antigen including but not limited to monoclonal antibodies, polyclonal antibodies, synthetic antibodies, scFvs, human antibodies, humanized antibodies, and fragments thereof.

With respect to the transmembrane domain, the CAR can be designed to comprise a transmembrane domain that is fused to the extracellular domain of the CAR. In one embodiment, the transmembrane domain that naturally is associated with one of the domains in the CAR is used. In some instances, the transmembrane domain can be selected or modified by amino acid substitution to avoid binding of such domains to the transmembrane domains of the same or different surface membrane proteins to minimize interactions with other members of the receptor complex.

The transmembrane domain may be derived either from a natural or from a synthetic source. Where the source is natural, the domain may be derived from any membrane-bound or transmembrane protein. Transmembrane regions of particular use in this invention may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker. The cytoplasmic domain or otherwise the intracellular signaling domain of the CAR of the invention is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR of the invention include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence: those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the invention include those derived from TCR zeta, FcR gamma, FcR beta, CD3 gamma, CD3 delta, CD3 epsilon, CD5, CD22, CD79a, CD79b, and CD66d. It is particularly preferred that cytoplasmic signaling molecule in the CAR of the invention comprises a cytoplasmic signaling sequence derived from CD3 zeta.

In a preferred embodiment, the cytoplasmic domain of the CAR can be designed to comprise the CD3-zeta signaling domain by itself or combined with any other desired cytoplasmic domain(s) useful in the context of the CAR of the invention. For example, the cytoplasmic domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such molecules include CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, and a ligand that specifically binds with CD83, and the like. Thus, while the invention in exemplified primarily with 4-1BB as the co-stimulatory signaling element, other costimulatory elements are within the scope of the invention.

The cytoplasmic signaling sequences within the cytoplasmic signaling portion of the CAR of the invention may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the cytoplasmic domain is designed to comprise the signaling domain of CD3-zeta and the signaling domain of CD28. In yet another embodiment, the cytoplasmic domain is designed to comprise the signaling domain of CD3-zeta and the signaling domain of CD28 and 4-1BB.

In one embodiment of the invention CAR-T cells are generated concurrently with lentiviral mediated silencing of NR2F6. Numerous means of generating CAR-T cells are known in the art. In one embodiment of the invention FMC63-28z CAR (Genebank identifier HM852952.1), is used as the template for the CAR except the anti-CD19, single-chain variable fragment sequence is replaced with an ROBO-4 fragment. The construct is synthesized and inserted into a pLNCX retroviral vector. Retroviruses encoding the ROBO-4-specific CAR are generated using the retrovirus packaging kit, Ampho (Takara), following the manufacturer's protocol. For generation of CAR-T cells donor blood is obtained and after centrifugation on Ficoll-Hypaque density gradients (Sigma-Aldrich), PBMCs are plated at 2×10(6) cells/mL in cell culture for 2 hours and the non-adherent cells are collected. The cells were then stimulated for 2 days on a non-tissue-culture-treated 24-well plate coated with 1 μg/mL OKT3 (Biolegend) at 1×10(6) cells/mL and in the presence of 1 μg/mL of anti-human CD28 antibody (Biolegend). For retrovirus transduction, a 24-well plate are coated with RetroNectin (Takara) at 4° C. overnight, according to the manufacturer's protocol, and then blocked with 2% BSA at room temperature for 30 min. The plate was then loaded with retrovirus supernatants at 300 μL/well and incubated at 37° C. for 6 h. Next, 1×10(6) stimulated PBLs in 1 mL of medium are added to 1 mL of retrovirus supernatants before being transferred to the pre-coated wells and cultured at 37° C. for 2 d. The cells are then transferred to a tissue-culture-treated plate at 1×10 (6)cells/mL and cultured in the presence of 100 U/mL of recombinant human IL-2 [1]. Other antigens may be used to replace ROBO-4 and these include: a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; l) legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globoll; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak)HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

Other means of generating CARs are known in the art and incorporated by reference. For example, Groner's group genetically modified T lymphocytes and endowed them with the ability to specifically recognize cancer cells. Tumor cells overexpressing the ErbB-2 receptor served as a model. The target cell recognition specificity was conferred to T lymphocytes by transduction of a chimeric gene encoding the zeta-chain of the TCR and a single chain antibody (scFv(FRP5)) directed against the human ErbB-2 receptor. The chimeric scFv(FRP5)-zeta gene was introduced into primary mouse T lymphocytes via retroviral gene transfer. Naive T lymphocytes were activated and infected by cocultivation with a retrovirus-producing packaging cell line. The scFv(FRP5)-zeta fusion gene was expressed in >75% of the T cells. These T cells lysed ErbB-2-expressing target cells in vitro with high specificity. In a syngeneic mouse model, mice were treated with autologous, transduced T cells. The adoptively transferred scFv(FRP5)-zeta-expressing T cells caused total regression of ErbB-2-expressing tumors. The presence of the transduced T lymphocytes in the tumor tissue was monitored. No humoral response directed against the transduced T cells was observed. Abs directed against the ErbB-2 receptor were detected upon tumor lysis [2]. Hombach et al. constructed an anti-CEA chimeric receptor whose extracellular moiety is composed of a humanized scFv derived from the anti-CEA mAb BW431/26 and the CH2/CH3 constant domains of human IgG. The intracellular moiety consists of the gamma-signaling chain of the human Fc epsilon RI receptor constituting a completely humanized chimeric receptor. After transfection, the humBW431/26 scFv-CH2CH3-gamma receptor is expressed as a homodimer on the surface of MD45 T cells. Co-incubation with CEA+ tumor cells specifically activates grafted MD45 T cells indicated by IL-2 secretion and cytolytic activity against CEA+ tumor cells. Notably, the efficacy of receptor-mediated activation is not affected by soluble CEA up to 25 micrograms/ml demonstrating the usefulness of this chimeric receptor for specific cellular activation by membrane-bound CEA even in the presence of high concentrations of CEA, as found in patients during progression of the disease [3]. These methods are described to guide one of skill in the art to practicing the invention, which in one embodiment is the utilization of CAR T cell approaches towards targeting tumor endothelium as comparted to simply targeting the tumor itself.

Targeting of mucins associated with cancers has been performed with CAR T cells by grafting the antibody that binds to the mucin with CD3 zeta chain. In an older publication chimeric immune receptor consisting of an extracellular antigen-binding domain derived from the CC49 humanized single-chain antibody, linked to the CD3zeta signaling domain of the T cell receptor, was generated (CC49-zeta). This receptor binds to TAG-72, a mucin antigen expressed by most human adenocarcinomas. CC49-zeta was expressed in CD4+ and CD8+ T cells and induced cytokine production on stimulation. Human T cells expressing CC49-zeta recognized and killed tumor cell lines and primary tumor cells expressing TAG-72. CC49-zeta T cells did not mediate bystander killing of TAG-72-negative cells. In addition, CC49-zeta T cells not only killed FasL-positive tumor cells in vitro and in vivo, but also survived in their presence, and were immunoprotective in intraperitoneal and subcutaneous murine tumor xenograft models with TAG-72-positive human tumor cells. Finally, receptor-positive T cells were still effective in killing TAG-72-positive targets in the presence of physiological levels of soluble TAG-72, and did not induce killing of TAG-72-negative cells under the same conditions [4].

For clinical practice of the invention several reports exist in the art that would guide the skilled artisan as to concentrations, cell numbers, and dosing protocols useful. While in the art CAR T cells have been utilized targeting surface tumor antigens, the main issue with this approach is the difficulty of T cells to enter tumors due to features specific to the tumor microenvironment. These include higher interstitial pressure inside the tumor compared to the surroundings [5-18], acidosis inside the tumor [19-39], and expression in the tumor of FasL which kills activated T cells [40-49]. Accordingly the invention seeks to more effectively utilize CAR T cells by directly targeting them to tumor endothelium, which is in direct contact with blood and therefore not susceptible to intratumoral factors the limit efficacy of conventional T cell therapies.

In one embodiment of the invention, protocols similar to Kershaw et al are utilized with the exception that tumor endothelial antigens are targeted as opposed to conventional tumor antigens. Such tumor endothelial antigens include CD93, TEM-1, VEGFR1, and survivin. Antibodies can be made for these proteins, methodologies for which are described in U.S. Pat. Nos. 5,225,539, 5,585,089, 5,693,761, and 5,639,641. In one example that may be utilized as a template for clinical development, T cells with reactivity against the ovarian cancer-associated antigen alpha-folate receptor (FR) were generated by genetic modification of autologous T cells with a chimeric gene incorporating an anti-FR single-chain antibody linked to the signaling domain of the Fc receptor gamma chain. Patients were assigned to one of two cohorts in the study. Eight patients in cohort 1 received a dose escalation of T cells in combination with high-dose interleukin-2, and six patients in cohort 2 received dual-specific T cells (reactive with both FR and allogeneic cells) followed by immunization with allogeneic peripheral blood mononuclear cells. Five patients in cohort 1 experienced some grade 3 to 4 treatment-related toxicity that was probably due to interleukin-2 administration, which could be managed using standard measures. Patients in cohort 2 experienced relatively mild side effects with grade 1 to 2 symptoms. No reduction in tumor burden was seen in any patient. Tracking 111In-labeled adoptively transferred T cells in cohort 1 revealed a lack of specific localization of T cells to tumor except in one patient where some signal was detected in a peritoneal deposit. PCR analysis showed that gene-modified T cells were present in the circulation in large numbers for the first 2 days after transfer, but these quickly declined to be barely detectable 1 month later in most patients [50]. Similar CAR-T clinical studies have been reported for neuroblastoma [51, 52], B cell malignancies [53-65], melanoma [66], ovarian cancer [67], renal cancer [68], mesothelioma [69], and head and neck cancer [70].

In one embodiment of the invention PBMCs are derived from leukapheresis and stimulated with anti-CD3 (OKT3, Ortho Biotech, Raritan, N.J.) and human recombinant IL-2 (600 IU/mL; Chiron, Emeryville, Calif.). After 3 days of culture, ˜5×10⁷ to 1×10⁸ lymphocytes are taken and transduced with retroviral vector supernatant (Cell Genesys, San Francisco, Calif.) encoding the chimeric CAR T recognizing tumor-endothelium specific antigen and subsequently selected for gene integration by culture in G418. In another embodiment the generation of dual-specific T cells is performed, stimulation of T cells is achieved by coculture of patient PBMCs with irradiated (5,000 cGy) allogeneic donor PBMCs from cryopre-served apheresis product (mixed lymphocyte reaction). The MHC haplotype of allogeneic donors is determined before use, and donors that differed in at least four MHC class I alleles from the patient are used. Culture medium consisted of AimV medium (Invitrogen, Carlsbad, Calif.) supplemented with 5% human AB⁻ serum (Valley Biomedical, Winchester, Va.), penicillin (50 units/mL), streptomycin (50 mg/mL; Bio Whittaker, Walkersville, Md.), amphotericin B (Fungizone, 1.25 mg/mL; Biofluids, Rockville, Md.), L-glutamine (2 mmol/L; Mediatech, Herndon, Va.), and human recombinant IL-2 (Proleukin, 300 IU/mL; Chiron). Mixed lymphocyte reaction consisted of 2×10⁶ patient PBMCs and 1×10⁷ allogeneic stimulator PBMCs in 2 mL AimV per well in 24-well plates. Between 24 and 48 wells are cultured per patient for 3 days, at which time transduction is done by aspirating 1.5 mL of medium and replacing with 2.0 mL retroviral supernatant containing 300 IU/mL IL-2, 10 mmol/L HEPES, and 8 μg/mL polybrene (Sigma, St. Louis, Mo.) followed by covering with plastic wrap and centrifugation at 1,000×g for 1 hour at room temperature. After overnight culture at 37° C./5% CO₂, transduction is repeated on the following day, and then medium was replaced after another 24 hours. Cells are then resuspended at 1×10⁶/mL in fresh medium containing 0.5 mg/mL G418 (Invitrogen) in 175-cm² flasks for 5 days before resuspension in media lacking G418. Cells are expanded to 2×10⁹ and then restimulated with allogeneic PBMCs from the same donor to enrich for T cells specific for the donor allogeneic haplotype. Restimulation is done by incubating patient T cells (1×10⁶/mL) and stimulator PBMCs (2×10⁶/mL) in 3-liter Fenwall culture bags in AimV+additives and IL-2 (no G418). Cell numbers were adjusted to 1×10⁶/mL, and IL-2 was added every 2 days, until sufficient numbers for treatment were achieved.

The present invention relates to the specific silencing of NR2F6 to augment CAR T cell. In one embodiment the present invention relates generally to the use of T cells genetically modified to stably express a desired CAR that possesses high affinity towards tumor associated endothelium or tumor antigens, while concurrently possessing reduced NR2F6 activity. Preferably, the cell can be genetically modified to stably express an antibody binding domain on its surface, conferring novel antigen specificity that is MHC independent. In some instances, the T cell is genetically modified to stably express a CAR that combines an antigen recognition domain of a specific antibody with an intracellular domain of the CD3-zeta chain or Fc.gamma.RI protein into a single chimeric protein. In one embodiment, the CAR of the invention comprises an extracellular domain having an antigen recognition domain, a transmembrane domain, and a cytoplasmic domain. In one embodiment, the transmembrane domain that naturally is associated with one of the domains in the CAR is used. In another embodiment, the transmembrane domain can be selected or modified by amino acid substitution to avoid binding of such domains to the transmembrane domains of the same or different surface membrane proteins to minimize interactions with other members of the receptor complex. Preferably, the transmembrane domain is the CD8.alpha. hinge domain. With respect to the cytoplasmic domain, the CAR of the invention can be designed to comprise the CD28 and/or 4-1BB and/or CD40 and/or OX40 signaling domain by itself or be combined with any other desired cytoplasmic domain(s) useful in the context of the CAR of the invention. In one embodiment, the cytoplasmic domain of the CAR can be designed to further comprise the signaling domain of CD3-zeta. For example, the cytoplasmic domain of the CAR can include but is not limited to CD3-zeta, 4-1BB and CD28 signaling modules and combinations thereof. In another embodiment of the invention inhibition of CTLA-4 is performed either by transfection with an shRNA possessing selectively towards CTLA-4 or by constructing the CAR to possess a dominant negative mutant of CTLA-4. This would render the CAR T cell resistant to inhibitory activities of the tumors. Accordingly, the invention provides CAR T cells and methods of their use for adoptive therapy. In one embodiment, the CAR T cells of the invention can be generated by introducing a lentiviral vector comprising a desired CAR, for example a CAR comprising anti-CD19, CD8.alpha. hinge and transmembrane domain, and human 4-1BB and CD3zeta signaling domains, into the cells. The CAR T cells of the invention are able to replicate in vivo resulting in long-term persistence that can lead to sustained tumor control.

One embodiment of the invention is a short-interfering ribonucleic acid (siRNA) molecule effective at silencing NR2F6 expression or substantially inhibiting NR2F6 expression that is administered to CAR-T cells or CAR-NK cells during their generation. In one embodiment of the invention the oligonucleotide backbone is chemically modified to increase the deliverability of the interfering ribonucleic acid molecule. In another embodiment these chemical modifications act to neutralize the negative charge of the interfering ribonucleic acid molecule. One embodiment of the invention consists of a pharmaceutical composition comprising an siRNA oligonucleotide that induces RNA interference against NR2F6. It is known to one of skill in the art that siRNAs induce a sequence-specific reduction in expression of a gene by the process of RNAi, as previously mentioned. Thus, siRNA is the intermediate effector molecule of the RNAi process that is normally induced by double stranded viral infections, with the longer double stranded RNA being cleaved by naturally occurring enzymes such as DICER. Some nucleic acid molecules or constructs provided herein include double stranded RNA molecules comprising 16-30, e.g., 16, 17, 18, 19, 20, 21, 22, 23, 24, 25, 26, 27, 28, 29, or 30 nucleotides in each strand, wherein one of the strands is substantially identical, for example at least 85% (or more, as for example, 90%, 95%, or 100%) identical, e.g., having 3, 2, 1, or 0 mismatched nucleotide(s), to a target region in the mRNA of NR2F6 and the other strand is identical or substantially identical to the first strand. However, it will be appreciated that the dsRNA molecules may have any number of nucleotides in each strand which allows them to reduce the level of NR2F6 protein, or the level of a nucleic acid encoding NR2F6. The dsRNA molecules provided herein can be chemically synthesized, or can be transcribed in vitro from a DNA template, or in vivo from, e.g., shRNA, which is mentioned below. The dsRNA molecules can be designed using any method known in the art. In one embodiment, CAR-T cells and/or CAR-NK cells are treated with nucleic acids provided herein can include both unmodified siRNAs and modified siRNAs as known in the art. For example, in some embodiments, siRNA derivatives can include siRNA having two complementary strands of nucleic acid, such that the two strands are crosslinked. For a specific example, a 3′ OH terminus of one of the strands can be modified, or the two strands can be crosslinked and modified at the 3′ OH terminus. The siRNA derivative can contain a single crosslink (one example of a useful crosslink is a psoralen crosslink). In some embodiments, the siRNA derivative has at its 3′ terminus a biotin molecule (for example, a photocleavable molecule such as biotin), a peptide (as an example an HIV Tat peptide), a nanoparticle, a peptidomimetic, organic compounds, or dendrimer. Modifying siRNA derivatives in this way can improve cellular uptake or enhance cellular targeting activities of the resulting siRNA derivative as compared to the corresponding siRNA, are useful for tracing the siRNA derivative in the cell, or improve the stability of the siRNA derivative compared to the corresponding siRNA.

The nucleic acids described within the practice of the current invention can include nucleic acids that are unconjugated or can be conjugated to another moiety, such as a nanoparticle, to enhance a desired property of the pharmaceutical composition. Properties useful in the development of a therapeutic agent include: a) absorption; b) efficacy; c) bioavailability; and d) half life in blood or in vivo. RNAi is believed to progress via at least one single stranded RNA intermediate, the skilled artisan will appreciate that single stranded-siRNAs (e.g., the antisense strand of a ds-siRNA) can also be designed as described herein and utilized according to the claimed methodologies.

In one embodiment the pharmaceutical composition comprises a nucleic acid-lipid particle that contains an siRNA oligonucleotide that induces RNA interference against NR2F6. in some aspects the lipid portion of the particle comprises a cationic lipid and a non-cationic lipid. In some aspects the nucleic acid-lipid particle further comprises a conjugated lipid that prevents aggregation of the particles and/or a sterol (e.g., cholesterol).

For practice of the invention, methods for expressing siRNA duplexes within cells from recombinant DNA constructs to allow longer-term target gene suppression in cells are known in the art, including mammalian Pol III promoter systems (e.g., H1 or U6/snRNA promoter systems) capable of expressing functional double-stranded siRNAs. Transcriptional termination by RNA Pol III occurs at runs of four consecutive T residues in the DNA template, providing a mechanism to end the siRNA transcript at a specific sequence. The siRNA is complementary to the sequence of the target gene in 5′-3′ and 3′-5′ orientations, and the two strands of the siRNA can be expressed in the same construct or in separate constructs. Hairpin siRNAs, driven by an H1 or U6 snRNA promoter can be expressed in cells, and can inhibit target gene expression. Constructs containing siRNA sequence(s) under the control of a T7 promoter also make functional siRNAs when co-transfected into the cells with a vector expressing T7 RNA polymerase. A single construct may contain multiple sequences coding for siRNAs, such as multiple regions of the NR2F6 gene, such as a nucleic acid encoding the NR2F6 mRNA, and can be driven, for example, by separate Pol III promoter sites. In some situations it will be preferable to induce expression of the hairpin siRNA or shRNAs in a tissue specific manner, in this case being T cells, in order to activate the shRNA transcription that would subsequently silence NR2F6 expression. Tissue specificity may be obtained by the use of regulatory sequences of DNA that are activated only in the desired tissue. Regulatory sequences include promoters, enhancers and other expression control elements such as polyadenylation signals. Regulatory sequences include those which direct constitutive expression of a nucleotide sequence in many types of host cell and those which direct expression of the nucleotide sequence only in certain host cells. Tissue specific promoters may be used to effect transcription in specific tissues or cells so as to reduce potential toxicity or undesirable effects to non-targeted tissues.

In one embodiment of the invention allogeneic T cells are used as a source of CAR-T cells for manipulation by silencing of NR2F6 or its analogues. Specific means of utilizing allogeneic CAR-T require the reduction of immunogenicity. Said reduction of immunogenicity may be accomplished by suppressing of HLA. Said suppression may be accomplished by a variety of means including administration of antisense oligonucleotides or RNA interference inducing molecules to said CAR-T.

In some embodiments, the CAR-target binding domain of the chimeric receptor protein comprises the antigen-binding portion of an immunoglobulin wherein the immunoglobulin binds a protein on the surface of the diseased cell. The antigen binding domain can be any domain that binds to the cell surface antigen including but not limited to ligands to the receptor or immunoglobulin proteins such as monoclonal antibodies, polyclonal antibodies, synthetic antibodies, human antibodies, humanized antibodies, and fragments thereof. In preferred embodiments, the antigen-binding domain of the CAR is constructed from the variable domains of an antibody that is able to specifically bind the antigen when part of a CAR construct. In some instances, it is beneficial for the antigen binding domain to be derived from the same species in which the CAR will ultimately be used in. For example, for use in humans, it may be beneficial for the antigen binding domain of the CAR to comprise a fragment of a human or humanized antibody. Accordingly, in some embodiments, the antigen binding domain portion of a CAR comprises a tumor antigen binding fragment of a human or humanized antibody. In each of these embodiments, the antigen-binding domain of an antibody, such as the single-chain variable fragment (scFV) or an Fab fragment or is fused to a transmembrane domain and a signaling intracellular domain (endodomain) of a T cell receptor. Often, a spacer or hinge is introduced between the extracellular antigen binding domain and the transmembrane domain to provide flexibility which allows the antigen-binding domain to orient in different directions to facilitate antigen recognition and binding. In some embodiments, the antigen binding moiety portion of the chimeric antigen T cell receptor targets the CEA antigen and comprises the CEA-binding domain of an antibody which has been shown to bind CEA expressed on a cell surface. The chimeric receptor construct can be generated according to methods and compositions known to the ordinarily skilled artisan. For example, a CEA CAR-T construct used in the Examples below comprises portions of the variable domain of a humanized MN14 antibody (described in U.S. Pat. No. 5,874,540, the contents of which are incorporated herein by reference it their entirety). A Fab or scFv construct can be generated from a CEA antibody according to the methods of Nolan et al. (1999, Clinical Canc Res, 5:3928-3941) to include the CEA-binding domains of the CEA antibody. In these and other embodiments, the antigen binding domain is an antibody or an antigen-binding fragment thereof. In another embodiment, the antigen-binding fragment is a Fab or a scFv. In yet a further embodiment, the stromal cell antigen is expressed on a stromal cell present in a tumor microenvironment. In another embodiment, the tumor is a carcinoma. In an additional embodiment, the stromal cell antigen is fibroblast activation protein (FAP). In yet other embodiments, the costimulatory signaling region comprises the intracellular domain of a costimulatory molecule selected from the group consisting of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83, and any combination thereof.

In one embodiment, NR2F6 silencing in CAR-T is utilized to augment immunity targeting tumor microenvironment, or more specifically, the cells associated with supporting tumor growth. The invention relates to compositions and methods for targeting stromal cells in the treatment of cancer. Immunotherapy for cancer, whether adoptive T cell therapy, antibody- or vaccine-based, has to date been focused primarily on targeting antigens expressed by the neoplastic cells. It is now evident that other components including stromal cells, infiltrating inflammatory/immune cells, vasculature and extracellular matrix that comprise the tumor microenvironment, are also required for or promote tumor growth and metastasis and therefore present additional therapeutic targets. In one embodiment, the present invention comprises compositions that target fibroblast activation protein (FAP). FAP is a cell surface protease that is expressed on the vast majority of stromal cells in virtually all human carcinomas. In one embodiment, the present invention provides an antibody that specifically binds to FAP. In one embodiment, the present invention provides compositions comprising an anti-FAP antibody, or an FAP binding fragment thereof. Non-limiting Examples of compositions targeting FAP encompassed by the present invention include antibodies, immunoconjugates, antibody conjugates, vaccines, and chimeric antigen receptors (CARs) that target FAP. The present invention has certain advantages over prior art cancer treatments in that antibody conjugates can have limited tumor penetration and often induce an immune response in the host, and vaccination may lead to long lasting endogenous immunity to FAP. The present compositions, i.e., using an anti-FAP CAR T cell is designed to circumvent these limitations. Other antigens than FAP may be targeted and these include a) Fos-related antigen 1; b) LCK; c) FAP; d) VEGFR2; e) NA17; f) PDGFR-beta; g) PAP; h) MAD-CT-2; i) Tie-2; j) PSA; k) protamine 2; 1)legumain; m) endosialin; n) prostate stem cell antigen; o)carbonic anhydrase IX; p) STn; q) Page4; r) proteinase 3; s) GM3 ganglioside; t) tyrosinase; u) MART1; v) gp100; w) SART3; x) RGS5; y) SSX2; z) Globoll; aa) Tn; ab) CEA; ac) hCG; ad) PRAME; ae) XAGE-1; af) AKAP-4; ag) TRP-2; ah) B7H3; ai) sperm fibrous sheath protein; aj) CYP1B1; ak)HMWMAA; al) sLe(a); am) MAGE A1; an) GD2; ao) PSMA; ap) mesothelin; aq) fucosyl GM1; ar) GD3; as) sperm protein 17; at) NY-ESO-1; au) PAX5; av) AFP; aw) polysialic acid; ax) EpCAM; ay) MAGE-A3; az) mutant p53; ba) ras; bb) mutant ras; bc) NY-BR1; bd) PAX3; be) HER2/neu; bf) OY-TES1; bg) HPV E6 E7; bh) PLAC1; bi) hTERT; bj) BORIS; bk) ML-IAP; bl) idiotype of b cell lymphoma or multiple myeloma; bm) EphA2; bn) EGFRvIII; bo) cyclin B1; bp) RhoC; bq) androgen receptor; br) surviving; bs) MYCN; bt) wildtype p53; bu) LMP2; by) ETV6-AML; bw) MUC1; bx) BCR-ABL; by) ALK; bz) WT1; ca) ERG (TMPRSS2 ETS fusion gene); cb) sarcoma translocation breakpoint; cc) STEAP; cd) OFA/iLRP; and ce) Chondroitin sulfate proteoglycan 4 (CSPG4).

In one embodiment, CAR-T cells are generated by inhibition of NR2F6 while inducing clonal expansion of tumor-specific T cells. Additionally, the invention provides the use of NR2F6 silencing during generation of DC-CIK type killer cells. Said cells can be expanded in vitro in response to tumor antigens, or can be CARs that are genetically engineered and transfected, or a combination of both. In one embodiment cellular lysates of tumor cells, or tumor stem cells are loaded into dendritic cells. In one embodiment the invention provides a means of generating a population of cells with tumoricidal ability that are reactive, to which focus is added by subsequent peptide specific vaccination. The generation of cytotoxic lymphocytes may be performed, in one embodiment by extracted 50 ml of peripheral blood from a cancer patient and peripheral blood monoclear cells (PBMC) are isolated using the Ficoll Method. PBMC are subsequently resuspended in 10 ml AIM-V media and allowed to adhere onto a plastic surface for 2-4 hours. The adherent cells are then cultured at 37° C. in AIM-V media supplemented with 1,000 U/mL granulocyte-monocyte colony-stimulating factor and 500 U/mL IL-4 after non-adherent cells are removed by gentle washing in Hanks Buffered Saline Solution (HBSS). Half of the volume of the GM-CSF and IL-4 supplemented media is changed every other day. Immature DCs are harvested on day 7. In one embodiment said generated DC are used to stimulate T cell and NK cell tumoricidal activity by pulsing with autologous tumor lysate. Specifically, generated DC may be further purified from culture through use of flow cytometry sorting or magnetic activated cell sorting (MACS), or may be utilized as a semi-pure population. DC pulsed with tumor lysate may be added into said patient in need of therapy with the concept of stimulating NK and T cell activity in vivo, or in another embodiment may be incubated in vitro with a population of cells containing T cells and/or NK cells. In one embodiment DC are exposed to agents capable of stimulating maturation in vitro and rendering them resistant to tumor derived inhibitory compounds such as arginase byproducts. Specific means of stimulating in vitro maturation include culturing DC or DC containing populations with a toll like receptor agonist. Another means of achieving DC maturation involves exposure of DC to TNF-alpha at a concentration of approximately 20 ng/mL. In order to activate T cells and/or NK cells in vitro, cells are cultured in media containing approximately 1000 IU/ml of interferon gamma. Incubation with interferon gamma may be performed for the period of 2 hours to the period of 7 days. Preferably, incubation is performed for approximately 24 hours, after which T cells and/or NK cells are stimulated via the CD3 and CD28 receptors. One means of accomplishing this is by addition of antibodies capable of activating these receptors. In one embodiment approximately, 2 ug/ml of anti-CD3 antibody is added, together with approximately 1 ug/ml anti-CD28. In order to promote survival of T cells and NK cells, was well as to stimulate proliferation, a T cell/NK mitogen may be used. In one embodiment the cytokine IL-2 is utilized. Specific concentrations of IL-2 useful for the practice of the invention are approximately 500 u/mL IL-2. Media containing IL-2 and antibodies may be changed every 48 hours for approximately 8-14 days. In one particular embodiment DC are included to said T cells and/or NK cells in order to endow cytotoxic activity towards tumor cells. In a particular embodiment, inhibitors of caspases are added in the culture so as to reduce rate of apoptosis of T cells and/or NK cells. Generated cells can be administered to a subject intradermally, intramuscularly, subcutaneously, intraperitoneally, intraarterially, intravenously (including a method performed by an indwelling catheter), intratumorally, or into an afferent lymph vessel. The immune response of the patient treated with these cytotoxic cells is assessed utilizing a variety of antigens found in tumor endothelial cells. When cytotoxic or antibody, or antibody associated with complement fixation are recognized to be upregulated in the cancer patient, subsequent immunizations are performed utilizing peptides to induce a focusing of the immune response.

In another embodiment DC are generated from leukocytes of patients by leukopheresis. Numerous means of leukopheresis are known in the art. In one example, a Frenius Device (Fresenius Com.Tec) is utilized with the use of the MNC program, at approximately 1500 rpm, and with a P1Y kit. The plasma pump flow rates are adjusted to approximately 50 mL/min. Various anticoagulants may be used, for example ACD-A. The Inlet/ACD Ratio may be ranged from approximately 10:1 to 16:1. In one embodiment approximately 150 mL of blood is processed. The leukopheresis product is subsequently used for initiation of dendritic cell culture. In order to generate a peripheral blood mononuclear cells from leukopheresis product, mononuclear cells are isolated by the Ficoll-Hypaque density gradient centrifugation. Monocytes are then enriched by the Percoll hyperosmotic density gradient centrifugation followed by two hours of adherence to the plate culture. Cells are then centrifuged at 500 g to separate the different cell populations. Adherent monocytes are cultured for 7 days in 6-well plates at 2×106 cells/mL RMPI medium with 1% penicillin/streptomycin, 2 mM L-glutamine, 10% of autologous, 50 ng/mL GM-CSF and 30 ng/mL IL-4. On day 6 immature dendritic cells are pulsed with tumor lysates. Pulsing may be performed by incubation of lysates with dendritic cells, or may be generated by fusion of immature dendritic cells with tumor lysates cells. Means of generating hybridomas or cellular fusion products are known in the art and include electrical pulse mediated fusion, or stimulation of cellular fusion by treatment with polyethelyne glycol. On day 7, the immature DCs are then induced to differentiate into mature DCs by culturing for 48 hours with 30 ng/mL interferon gamma (IFN-γ). During the course of generating DC for clinical purposes, microbiologic monitoring tests are performed at the beginning of the culture, on the fifth day and at the time of cell freezing for further use or prior to release of the dendritic cells. Administration of tumor lysate pulsed dendritic cells is utilized as a polyvalent vaccine, whereas subsequent to administration antibody or t cell responses are assessed for induction of antigen specificity, peptides corresponding to immune response stimulated are used for further immunization to focus the immune response. NR2F6 silencing may be performed in the reacting lymphoid cells whether they be T cells, B cells, NK cells, NKT cells or gamma delta T cells.

In some embodiments, culture of the immune effectors cells is performed after extracting from a patient that has been immunized with a antigenic preparation. Said immature effectors are subsequently silenced for NR2F6. Specifically separating the cell population and cell sub-population containing a T cell can be performed, for example, by fractionation of a mononuclear cell fraction by density gradient centrifugation, or a separation means using the surface marker of the T cell as an index. Subsequently, isolation based on surface markers may be performed. Examples of the surface marker include CD3, CD8 and CD4, and separation methods depending on these surface markers are known in the art. For example, the step can be performed by mixing a carrier such as beads or a culturing container on which an anti-CD8 antibody has been immobilized, with a cell population containing a T cell, and recovering a CD8-positive T cell bound to the carrier. As the beads on which an anti-CD8 antibody has been immobilized, for example, CD8 MicroBeads), Dynabeads M450 CD8, and Eligix anti-CD8 mAb coated nickel particles can be suitably used. This is also the same as in implementation using CD4 as an index and, for example, CD4 MicroBeads, Dynabeads M-450 CD4 can also be used. In some embodiments of the invention, T regulatory cells are depleted before initiation of the culture. Depletion of T regulatory cells may be performed by negative selection by removing cells that express makers such as neuropilin, CD25, CD4, CTLA4, and membrane bound TGF-beta. Experimentation by one of skill in the art may be performed with different culture conditions in order to generate effector lymphocytes, or cytotoxic cells, that possess both maximal activity in terms of tumor killing, as well as migration to the site of the tumor. For example, the step of culturing the cell population and cell sub-population containing a T cell can be performed by selecting suitable known culturing conditions depending on the cell population. In addition, in the step of stimulating the cell population, known proteins and chemical ingredients, etc., may be added to the medium to perform culturing. For example, cytokines, chemokines or other ingredients may be added to the medium. Herein, the cytokine is not particularly limited as far as it can act on the T cell, and examples thereof include IL-2, IFN-.gamma., transforming growth factor (TGF)-.beta., IL-15, IL-7, IFN-.alpha., IL-12, CD40L, and IL-27. From the viewpoint of enhancing cellular immunity, particularly suitably, IL-2, IFN-.gamma., or IL-12 is used and, from the viewpoint of improvement in survival of a transferred T cell in vivo, IL-7, IL-15 or IL-21 is suitably used. In addition, the chemokine is not particularly limited as far as it acts on the T cell and exhibits migration activity, and examples thereof include RANTES, CCL21, MIP1.alpha., MIP1.beta., CCL19, CXCL12, IP-10 and MIG. The stimulation of the cell population can be performed by the presence of a ligand for a molecule present on the surface of the T cell, for example, CD3, CD28, or CD44 and/or an antibody to the molecule. Further, the cell population can be stimulated by contacting with other lymphocytes such as antigen presenting cells (dendritic cell) presenting a target peptide such as a peptide derived from a cancer antigen on the surface of a cell. In addition to assessing cytotoxicity and migration as end points, it is within the scope of the current invention to optimize the cellular product based on other means of assessing T cell activity, for example, the function enhancement of the T cell in the method of the present invention can be assessed at a plurality of time points before and after each step using a cytokine assay, an antigen-specific cell assay (tetramer assay), a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. In vivo assessment of the efficacy of the generated cells using the invention may be assessed in a living body before first administration of the T cell with enhanced function of the present invention, or at various time points after initiation of treatment, using an antigen-specific cell assay, a proliferation assay, a cytolytic cell assay, or an in vivo delayed hypersensitivity test using a recombinant tumor-associated antigen or an immunogenic fragment or an antigen-derived peptide. Examples of an additional method for measuring an increase in an immune response include a delayed hypersensitivity test, flow cytometry using a peptide major histocompatibility gene complex tetramer. a lymphocyte proliferation assay, an enzyme-linked immunosorbent assay, an enzyme-linked immunospot assay, cytokine flow cytometry, a direct cytotoxity assay, measurement of cytokine mRNA by a quantitative reverse transcriptase polymerase chain reaction, or an assay which is currently used for measuring a T cell response such as a limiting dilution method. Further, an immune response can be assessed by a weight, diameter or malignant degree of a tumor possessed by a living body, or the survival rate or survival term of a subject or group of subjects. Said cells can be expanded in the presence of specific antigens associated with tumor endothelium and subsequently injected into the patient in need of treatment. Within the context of the invention, teachings are provided to amplify an antigen specific immune response following immunization with a polyvalent vaccine, in which the antigenic epitopes are used for immunization together with adjuvants such as toll like receptors (TLRs). In vivo silencing of NR2F6 is performed using a small molecule inhibitor or RNA interference associated methods. These molecules are type 1 membrane receptors that are expressed on hematopoietic and non-hematopoietic cells. At least 11 members have been identified in the TLR family. These receptors are characterized by their capacity to recognize pathogen-associated molecular patterns (PAMP) expressed by pathogenic organisms. It has been found that triggering of TLR elicits profound inflammatory responses through enhanced cytokine production, chemokine receptor expression (CCR2, CCR5 and CCR7), and costimulatory molecule expression. As such, these receptors in the innate immune systems exert control over the polarity of the ensuing acquired immune response. Among the TLRs, TLR9 has been extensively investigated for its functions in immune responses. Stimulation of the TLR9 receptor directs antigen-presenting cells (APCs) towards priming potent, T.sub.H1-dominated T-cell responses, by increasing the production of pro-inflammatory cytokines and the presentation of co-stimulatory molecules to T cells. CpG oligonucleotides, ligands for TLR9, were found to be a class of potent immunostimulatory factors. CpG therapy has been tested against a wide variety of tumor models in mice, and has consistently been shown to promote tumor inhibition or regression.

In some embodiments of the invention, specific antigens are immunized following polyvalent immunization, said specific antigens administered in the form of DNA vaccines. Numerous publications have reported animal and clinical efficacy of DNA vaccines which are incorporated by reference [15-17]. In addition to direct DNA injection techniques, DNA vaccines can be administered by electroporation [18]. The nucleic acid compositions, including the DNA vaccine compositions, may further comprise a pharmaceutically acceptable excipient. Examples of suitable pharmaceutically acceptable excipients for nucleic acid compositions, including DNA vaccine compositions, are well known to those skilled in the art and include sugars, etc. Such excipients may be aqueous or non aqueous solutions, suspensions, and emulsions. Examples of non-aqueous excipients include propylene glycol, polyethylene glycol, vegetable oils such as olive oil, and injectable organic esters such as ethyl oleate. Examples of aqueous excipient include water, alcoholic/aqueous solutions, emulsions or suspensions, including saline and buffered media. Suitable excipients also include agents that assist in cellular uptake of the polynucleotide molecule. Examples of such agents are (i) chemicals that modify cellular permeability, such as bupivacaine, (ii) liposomes or viral particles for encapsulation of the polynucleotide, or (iii) cationic lipids or silica, gold, or tungsten microparticles which associate themselves with the polynucleotides. Anionic and neutral liposomes are well-known in the art (see, e.g., Liposomes: A Practical Approach, RPC New Ed, IRL press (1990), for a detailed description of methods for making liposomes) and are useful for delivering a large range of products, including polynucleotides. Cationic lipids are also known in the art and are commonly used for gene delivery. Such lipids include Lipofectin™ also known as DOTMA (N-[I-(2,3-dioleyloxy) propyls N,N, N-trimethylammonium chloride), DOTAP (1,2-bis (oleyloxy)-3 (trimethylammonio) propane), DDAB (dimethyldioctadecyl-ammonium bromide), DOGS (dioctadecylamidologlycyl spermine) and cholesterol derivatives such as DCChol (3 beta-(N-(N′,N′-dimethyl aminomethane)-carbamoyl) cholesterol). A description of these cationic lipids can be found in EP 187,702, WO 90/11092, U.S. Pat. No. 5,283,185, WO 91/15501, WO 95/26356, and U.S. Pat. No. 5,527,928. A particular useful cationic lipid formulation that may be used with the nucleic vaccine provided by the disclosure is VAXFECTIN, which is a commixture of a cationic lipid (GAP-DMORIE) and a neutral phospholipid (DPyPE) which, when combined in an aqueous vehicle, self-assemble to form liposomes. Cationic lipids for gene delivery are preferably used in association with a neutral lipid such as DOPE (dioleyl phosphatidylethanolamine), as described in WO 90/11092 as an example. In addition, a DNA vaccine can also be formulated with a nonionic block copolymer such as CRL1005. Other immunization means include prime boost regiments [19]. The polypeptide and nucleic acid compositions can be administered to an animal, including human, by a number of methods known in the art. Examples of suitable methods include: (1) intramuscular, intradermal, intraepidermal, intravenous, intraarterial, subcutaneous, or intraperitoneal administration, (2) oral administration, and (3) topical application (such as ocular, intranasal, and intravaginal application). One particular method of intradermal or intraepidermal administration of a nucleic acid vaccine composition that may be used is gene gun delivery using the Particle Mediated Epidermal Delivery (PMED™) vaccine delivery device marketed by PowderMed [20]. PMED is a needle-free method of administering vaccines to animals or humans. The PMED system involves the precipitation of DNA onto microscopic gold particles that are then propelled by helium gas into the epidermis [21]. The DNA-coated gold particles are delivered to the APCs and keratinocytes of the epidermis, and once inside the nuclei of these cells, the DNA elutes off the gold and becomes transcriptionally active, producing encoded protein. This protein is then presented by the APCs to the lymphocytes to induce a T-cell-mediated immune response. Another particular method for intramuscular administration of a nucleic acid vaccine provided by the present disclosure is electroporation [22]. Electroporation uses controlled electrical pulses to create temporary pores in the cell membrane, which facilitates cellular uptake of the nucleic acid vaccine injected into the muscle [23-26]. Where a CpG is used in combination with a nucleic acid vaccine, it is preferred that the CpG and nucleic acid vaccine are co-formulated in one formulation and the formulation is administered intramuscularly by electroporation. A helper T cell and cytotoxic T cell stimulatory polypeptide can be introduced into a mammalian host, including humans, linked to its own carrier or as a homopolymer or heteropolymer of active polypeptide units. Such a polymer can elicit increase immunological reaction and, where different polypeptides are used to make up the polymer, the additional ability to induce antibodies and/or T cells that react with different antigenic determinants of the tumor. Useful carriers known in the art include, for example, thyroglobulin, albumins such as human serum albumin, tetanus toxoid, polyamino acids such as poly(D-lysine:D-glutamic acid), influenza polypeptide, and the like. Adjuvants such as incomplete Freunds adjuvant, GM-CSF, aluminum phosphate, CpG containing DNA, inulin, Poly (IC), aluminum hydroxide, alum, or montanide can also be used in the administration of an helper T cell and cytotoxic T cell stimulatory polypeptide.

Administration of the NR2F6 silenced CAR-T of the invention to a human patient can be by any route, including but not limited to intravenous, intradermal, transdermal, subcutaneous, intramuscular, inhalation (e.g., via an aerosol), buccal (e.g., sub-lingual), topical (i.e., both skin and mucosal surfaces, including airway surfaces), intrathecal, intraarticular, intraplural, intracerebral, intra-arterial, intraperitoneal, oral, intralymphatic, intranasal, rectal or vaginal administration, by perfusion through a regional catheter, or by direct intralesional injection. In a preferred embodiment, the compositions of the invention are administered by intravenous push or intravenous infusion given over defined period (e.g., 0.5 to 2 hours). The compositions of the invention can be delivered by peristaltic means or in the form of a depot, although the most suitable route in any given case will depend, as is well known in the art, on such factors as the species, age, gender and overall condition of the subject, the nature and severity of the condition being treated and/or on the nature of the particular composition (i.e., dosage, formulation) that is being administered. In particular embodiments, the route of administration is via bolus or continuous infusion over a period of time, once or twice a week. In other particular embodiments, the route of administration is by subcutaneous injection given in one or more sites (e.g. thigh, waist, buttocks, arm), optionally once or twice weekly. In one embodiment, the compositions, and/or methods of the invention are administered on an outpatient basis. Those skilled in the art will appreciate that dosages can be selected based on a number of factors including the age, sex, species and condition of the subject (e.g., activity of autoimmune disease or disorder), the desired degree of cellular or autoimmune antibody depletion, the disease to be treated and/or the particular antibody or antigen-binding fragment being used and can be determined by one of skill in the art. For example, effective amounts of the compositions of the invention may be extrapolated from dose-response curves derived from in vitro test systems or from animal model (e.g. the cotton rat or monkey) test systems. Models and methods for evaluation of the effects of antibodies are known in the art (Wooldridge et al., Blood, 89(8): 2994-2998 (1997), incorporated by reference herein in its entirety). In certain embodiments, for a particular disease or disorder, therapeutic regimens standard in the art for antibody therapy can be used with the compositions and methods of the invention. Examples of dosing regimens that can be used in the methods of the invention include, but are not limited to, daily, three times weekly (intermittent), weekly, or every 14 days. In certain embodiments, dosing regimens include, but are not limited to, monthly dosing or dosing every 6-8 weeks. Those skilled in the art will appreciate that dosages are generally higher and/or frequency of administration greater for initial treatment as compared with maintenance regimens.

### EXAMPLES

### Example 1: Detection of NR2F6 in T Cell Lines

Jurkat and HL-60 are obtained from American Type Tissue Culture (ATCC: Manassas, Va.) and grown under fully humidified 5% CO2 environment with DMEM supplemented with 10% FBS, 2% sodium pyruvate, non-essential amino acids (2 mM), penicillin (100 units/ml), streptomycin (100 μg/ml), and glutamine (4 mM) (Gibco-BRL). For some experiments NR2F6 induction will be achieved by pretreatment with anti-CD3/anti-CD28 beads.

Total RNA is isolated using the RNeasy Mini Kit (QIAGEN). Specifically, cells are trypsinized and harvested at a concentration of 5-10×106 cells, as a cell pellet after washing in PBS an appropriate volume of Buffer RLT Plus will be added and the cells will be vortexted for 30 seconds. This will result in lysis of the cells, with the lysate then being spun at 3 minutes at 15000 g. The supernatant is then removed and applied to a gDNA Eliminator spin column which is then placed in a 2 ml collection tube. Subsequently, the collected material is centrifuged for 30 s at ≥8000×g (≥10,000 rpm). The column is discarded and the flow-through is saved. The same volume (usually 350 μl or 600 μl) of 70% ethanol is added to the flow-through that has been collected. Up to 700 μl of the sample, including any precipitate, is then added to an RNeasy spin column placed in a 2 ml collection tube and the tube is centrifuged for 15 s at ≥8000×g. The flow-through is discarded. 700 μl of Buffer RW1 is then added to the RNeasy Mini spin column (in a 2 ml collection tube) and centrifuged for 15 s at ≥8000×g. 500 μl of Buffer RPE is added to the RNeasy spin column and centrifuged for 15 s at ≥8000×g. Subsequently 500 μl of Buffer RPE is added to the RNeasy spin column and centrifuged for 2 min at ≥8000×g (≥10,000 rpm). The RNeasy spin column will then be placed in a new 1.5 ml collection tube. Approximately 30-50 μl RNase-free water is added directly to the spin column membrane and centrifuged for 1 min at ≥8000×g to elute the RNA. Reverse transcription performed using Moloney murine leukemia virus reverse transcriptase (Promega) following the manufacturer's instructions. Reverse-transcribed products will be analyzed on a Mastercyler Ep Realplex (Eppendorf) using the QuantiFast SYBR Green PCR Kit (QIAGEN) according to the manufacturer's instructions.

As gene-specific primers, the following oligo-DNAs are assessed

Human NR2F6:
Fwd:
5′-TCTCCCAGCTGTTCTTCATGC-3′
Revs:
5′-CCAGTTGAAGGTACTCCCCG-3′
Human GAPDH:
Fwd:
5′-GGCCTCCAAGGAGTAAGACC-3′
Revs:
5′-AGGGGTCTACATGGCAACTG-3′.

As an internal control β-actin mRNA is also amplified using the following primers: β-actin forward, 5′-ATCTGGCACCACACCTTCTACAATGAGCTGCG-3′; β-actin reverse, 5′-CGTCATACTCCTGCTTGCTGATCCACATCTGC-3

PCR products will be size-separated on a 1.5% agarose gel; expression levels normalized to the GAPDH mRNA product, and will be visualized by SYBR Safe DNA gel staining (invitrogen)

NR2F6 transcript is found in Jurkat T cell lines

### Example 2: NR2F6 Silencing in T Cells

Jurkat and HL-60 cells either growing in stable conditions or pretreated with antiCD3/antiCD28 or 48 hours as used in Example 1.

To prepare the modified siRNA duplexes, complementary strands were mixed at equal concentrations, then heated at 70° for 1 min and allowed to anneal at 37° for 30 min. Successful annealing will be assessed with polyacrylamide gel electrophoresis.

Specific sequences tested are

(human siRNA)
Seq 1
GCCGUCUCAAGAAGUGCUU
(human siRNA)
Seq 2
CAUUGAGACACUGAUCAGA
(human siRNA)
Seq 3
GCAAGCAUUACGGUGUCUU
(human siRNA)
Seq 4
CCCCUAGCAUGAACUUGUG

Cells are transfected using the Amaxa Nucleofector Kit V (Amaxa biosystems, Koeln, Germany). Briefly, 3*10⁵ cells were resuspended in 100 μl of the nucleofector solution V and mixed with 1.5 μg of siRNA, then electroporated using program V005. Alternatively, lipofectamine based transfection may be utilized depending on efficacy.

Suppression of NR2F6 gene expression is observed following gene silencing.

### Example 3: Augmentation of T Cell Activity after NR2F6 Gene Silencing

PBMC are isolated by the following protocol:
1. Mix Ficoll-Paque PLUS thoroughly before using by inverting the bottom repeatedly.
2. Add Ficoll to tube. The amount depends on blood volume and tube size. For example, for 4 ml of blood, use 10 ml of Ficoll in a 50 ml tube. To this mixture is added the ingredient of step 3.
3. Dilute blood 2× with a phosphate buffered saline (PBS) plus 2 percent fetal bovine serum (i.e. PBS+2% FBS; or use other similarly suited culture medium). For the volume example listed above in step 2, 5 ml of PBS solution would be added.
4. Layer diluted blood on the top of the Ficoll solution. Be careful to minimize any mixing of blood with the Ficoll.
5. Centrifuge tube(s) at room temperature (i.e. 15-25° C.) for 30 minutes at 400×g, with the brake in the off position.
6. Remove and discard (or save for later use) the upper plasma layer carefully using a clean pipette so as not to disturb the remaining plasma-Ficoll interface solution. This is where the lymphocytes are found.
7. Using a clean pipette, transfer to a clean centrifuge tube the mononuclear/lymphocyte cell layer at the plasma-Ficoll interface. It is important to remove all the interface but a very minimum amount of Ficoll. Taking too much Ficoll will result in granulocyte contamination. Take care as well not to disturb the bottom erythrocyte-Ficoll pellet.
8. Add at least 3× volumes of balanced salt solution to the mononuclear/lymphocyte cells in the test tube.
9. Suspend the cells by drawing them in and out of a Pasteur pipette.
10. Centrifuge at 200×g for 10 minutes at room temperature. Steps 7-10 are important for removing any contaminating Ficoll and platelets/plasma proteins.
11. Repeat steps 8-10.

T cells are purified using MACs CD3 positive selection. Electroporation transfection method is performed according to manufacturer's instructions of 4D Nucleofector Device and Amaxa P3 primary cell 4D-Nucleofector X kit from Lonza. Some T were also transfected with pmax-GFPvector (1 μg/sample, Lonza) to interrogate the transfection efficiency. Transfection efficiency will be monitored by fluorescence microscopy and measured by flow cytometry at various time points. Gene silencing will be performed by real-time PCR. For T cell activation and expansion the following protocol is used:

Dynabeads® Washing Procedure

Dynabeads® should be washed before use.
1. Resuspend the Dynabeads® Human T-Activator CD3/CD28 in the vial.
2. Transfer the desired volume of Dynabeads® to a tube.
3. Add an equal volume of Buffer, or at least 1 ml, and mix (vortex for 5 seconds, or keep on a roller for at least 5 min).
4. Place the tube on a magnet for 1 min and discard the supernatant.
5. Remove the tube from the magnet and resuspend the washed Dynabeads® in the same volume of Culture Medium as the initial volume of Dynabeads® taken from the vial (step 2).

Activation of Human T Cells
1. Start with 8×10⁴ purified T cells in 100-200 μl medium in a 96-well tissue culture plate.
2. Add 2 μl Dynabeads® Human T-Activator CD3/CD28 to obtain a bead-to-cell ratio of 1:1.
3. Incubate in a humidified CO₂ incubator at 37° C., according to your specific experimental requirements.
4. Harvest the activated T cells and use directly for further analysis.
5. For flow cytometry applications, remove the beads prior to staining. Place the tube on a magnet for 1-2 minutes to separate the beads from the solution. Transfer the supernatant containing the cells to a new tube.

Expansion of Human T Cells
1. Start with 1-1.5×10⁶ purified T cells/ml in culture medium in a suitable tissue culture plate or tissue culture flask.
2. Add Dynabeads® Human T-Activator CD3/CD28 at a bead-to-cell ratio of 1:1.
3. Add 30 U/ml rIL-2.
4. Incubate in a humidified CO₂ incubator at 37° C., according to your specific experimental requirements.
5. Examine cultures daily, noting cell size and shape. Cell shrinking and reduced proliferation rate is typically observed in exhausted cell cultures.
6. Count the cells at least twice weekly after thorough re-suspension.
7. When the cell density exceeds 2.5×10⁶ cells/ml or when the medium turns yellow, split cultures back to a density of 0.5-1×10⁶ cells/ml in culture medium containing 30 U/ml rIL-2.

T cell activation, as assessed by proliferation and IFN-gamma production after antiCD3/antiCD28 stimulation is higher in NR2F6 silenced cells as compared to control silenced cells or anti-CTLA4 or anti-PD1 treated T cells.

### REFERENCES

1. Deng, Z., et al., Adoptive T-cell therapy of prostate cancer targeting the cancer stem cell antigen EpCAM. BMC Immunol, 2015. 16(1): p. 1.
2. Altenschmidt, U., E. Klundt, and B. Groner, Adoptive transfer of in vitro-targeted, activated T lymphocytes results in total tumor regression. J Immunol, 1997. 159(11): p. 5509-15.
3. Hombach, A., et al., A chimeric receptor that selectively targets membrane-bound carcinoembryonic antigen (mCEA) in the presence of soluble CEA. Gene Ther, 1999. 6(2): p. 300-4.
4. McGuinness, R. P., et al., Anti-tumor activity of human T cells expressing the CC49-zeta chimeric immune receptor. Hum Gene Ther, 1999. 10(2): p. 165-73.
5. Munson, J. M. and A. C. Shieh, Interstitial fluid flow in cancer: implications for disease progression and treatment. Cancer Manag Res, 2014. 6: p. 317-28.
6. Omidi, Y. and J. Barar, Targeting tumor microenvironment: crossing tumor interstitial fluid by multifunctional nanomedicines. Bioimpacts, 2014. 4(2): p. 55-67.
7. Wu, M., et al., The effect of interstitial pressure on therapeutic agent transport: coupling with the tumor blood and lymphatic vascular systems. J Theor Biol, 2014. 355: p. 194-207.
8. Sheth, R. A., et al., Barriers to drug delivery in interventional oncology. J Vasc Intery Radiol, 2013. 24(8): p. 1201-7.
9. Provenzano, P. P. and S. R. Hingorani, Hyaluronan, fluid pressure, and stromal resistance in pancreas cancer. Br J Cancer, 2013. 108(1): p. 1-8.
10. Grantab, R. H. and I. F. Tannock, Penetration of anticancer drugs through tumour tissue as a function of cellular packing density and interstitial fluid pressure and its modification by bortezomib. BMC Cancer, 2012. 12: p. 214.
11. Guo, P. and B. M. Fu, Effect of wall compliance and permeability on blood-flow rate in counter-current microvessels formed from anastomosis during tumor-induced angiogenesis. J Biomech Eng, 2012. 134(4): p. 041003.
12. Moen, I., et al., Hyperoxia increases the uptake of 5-fluorouracil in mammary tumors independently of changes in interstitial fluid pressure and tumor stroma. BMC Cancer, 2009. 9: p. 446.
13. Bhattacharya, A., et al., Inhibition of colon cancer growth by methylselenocysteine-induced angiogenic chemomodulation is influenced by histologic characteristics of the tumor. Clin Colorectal Cancer, 2009. 8(3): p. 155-62.
14. Nathan, S. S., et al., Elevated physiologic tumor pressure promotes proliferation and chemosensitivity in human osteosarcoma. Clin Cancer Res, 2005. 11(6): p. 2389-97.
15. Griffon-Etienne, G., et al., Taxane-induced apoptosis decompresses blood vessels and lowers interstitial fluid pressure in solid tumors: clinical implications. Cancer Res, 1999. 59(15): p. 3776-82.
16. Lee, I., Y. Boucher, and R. K. Jain, Nicotinamide can lower tumor interstitial fluid pressure: mechanistic and therapeutic implications. Cancer Res, 1992. 52(11): p. 3237-40.
17. Netti, P. A., et al., Enhancement of fluid filtration across tumor vessels: implication for delivery of macromolecules. Proc Natl Acad Sci USA, 1999. 96(6): p. 3137-42.
18. Tveit, E., R. Hultborn, and L. Weiss, Effects of noradrenaline on interstitial fluid pressure in induced rat mammary tumours. Cancer Lett, 1985. 27(3): p. 249-53.
19. Barar, J. and Y. Omidi, Dysregulated pH in Tumor Microenvironment Checkmates Cancer Therapy. Bioimpacts, 2013. 3(4): p. 149-62.
20. Chen, L. Q., et al., Evaluations of Tumor Acidosis Within In Vivo Tumor Models Using Parametric Maps Generated with AcidoCEST MRI. Mol Imaging Biol, 2015.
21. El Imad, T., L. El Khoury, and A. S. Geara, Warburg's effect on solid tumors. Saudi J Kidney Dis Transpl, 2014. 25(6): p. 1270-7.
22. Fukamachi, T., et al., Expression of acidosis-dependent genes in human cancer nests. Mol Clin Oncol, 2014. 2(6): p. 1160-1166.
23. Luo, Z., et al., Widefield optical imaging of changes in uptake of glucose and tissue extracellular pH in head and neck cancer. Cancer Prev Res (Phila), 2014. 7(10): p. 1035-44.
24. Pastorek, J. and S. Pastorekova, Hypoxia-induced carbonic anhydrase IX as a target for cancer therapy: From biology to clinical use. Semin Cancer Biol, 2014.
25. Adochite, R. C., et al., Targeting breast tumors with pH (low) insertion peptides. Mol Pharm, 2014. 11(8): p. 2896-905.
26. Visioli, F., et al., Glucose-regulated protein 78 (Grp78) confers chemoresistance to tumor endothelial cells under acidic stress. PLoS One, 2014. 9(6): p. e101053.
27. Ditte, Z., et al., Carnosine inhibits carbonic anhydrase IX-mediated extracellular acidosis and suppresses growth of HeLa tumor xenografts. BMC Cancer, 2014. 14: p. 358.
28. Thews, O., et al., Impact of hypoxia-related tumor acidosis on cytotoxicity of different chemotherapeutic drugs in vitro and in vivo. Adv Exp Med Biol, 2014. 812: p. 51-8.
29. Riemann, A., et al., Acidic priming enhances metastatic potential of cancer cells. Pflugers Arch, 2014. 466(11): p. 2127-38.
30. Federici, C., et al., Exosome release and low pH belong to a framework of resistance of human melanoma cells to cisplatin. PLoS One, 2014. 9(2): p. e88193.
31. Sedlakova, O., et al., Carbonic anhydrase IX, a hypoxia-induced catalytic component of the pH regulating machinery in tumors. Front Physiol, 2014. 4: p. 400.
32. Damaghi, M., J. W. Wojtkowiak, and R. J. Gillies, pH sensing and regulation in cancer. Front Physiol, 2013. 4: p. 370.
33. Justus, C. R., L. Dong, and L. V. Yang, Acidic tumor microenvironment and pH-sensing G protein-coupled receptors. Front Physiol, 2013. 4: p. 354.
34. Sugiyama, Y., et al., The heme precursor 5-aminolevulinic acid disrupts the Warburg effect in tumor cells and induces caspase-dependent apoptosis. Oncol Rep, 2014. 31(3): p. 1282-6.
35. Navratilova, J., et al., Acidic pH of tumor microenvironment enhances cytotoxicity of the disulfiram/Cu2+ complex to breast and colon cancer cells. Chemotherapy, 2013. 59(2): p. 112-20.
36. Peppicelli, S., et al., Acidic pH via NF-kappaB favours VEGF-C expression in human melanoma cells. Clin Exp Metastasis, 2013. 30(8): p. 957-67.
37. Han, L., et al., Acid active receptor-specific peptide ligand for in vivo tumor-targeted delivery. Small, 2013. 9(21): p. 3647-58.
38. Weerakkody, D., et al., Family of pH (low) insertion peptides for tumor targeting. Proc Natl Acad Sci USA, 2013. 110(15): p. 5834-9.
39. Parks, S. K., et al., Hypoxia promotes tumor cell survival in acidic conditions by preserving ATP levels. J Cell Physiol, 2013. 228(9): p. 1854-62.
40. Fang, L., et al., Effects of FasL expression in oral squamous cell cancer. Asian Pac J Cancer Prev, 2013. 14(1): p. 281-5.
41. Hasby, E. A., Weapons ovarian epithelial tumors may use in immune escape: an immunohistochemical correlational study. Pathol Oncol Res, 2012. 18(2): p. 509-18.
42. LA, O. R., et al., Membrane-bound Fas ligand only is essential for Fas-induced apoptosis. Nature, 2009. 461(7264): p. 659-63.
43. Zielinska, K., et al., Fas and FasL expression on cells of two transplantable melanoma lines according to their different biological properties. Folia Histochem Cytobiol, 2008. 46(3): p. 337-43.
44. Yoshikawa, T., et al., Elevated Fas expression is related to increased apoptosis of circulating CD8+ T cell in patients with gastric cancer. J Surg Res, 2008. 148(2): p. 143-51.
45. Xu, G. and J. Zhang, Suppression of FasL expression in tumor cells and preventing TNF-induced apoptosis was better for immune cells survival. J Cancer Res Clin Oncol, 2008. 134(10): p. 1043-9.
46. Das, T., et al., Renal cell carcinoma tumors induce T cell apoptosis through receptor-dependent and receptor-independent pathways. J Immunol, 2008. 180(7): p. 4687-96.
47. Zhang, J. and G. Xu, Suppression of FasL expression in tumor cells and preventing tumor necrosis factor-induced apoptosis by adenovirus 14.7K is an effective escape mechanism for immune cells. Cancer Genet Cytogenet, 2007. 179(2): p. 112-7.
48. Kassouf, N. and M. H. Thornhill, Oral cancer cell lines can use multiple ligands, including Fas-L, TRAIL and TNF-alpha, to induce apoptosis in Jurkat T cells: possible mechanisms for immune escape by head and neck cancers. Oral Oncol, 2008. 44(7): p. 672-82.
49. Wada, A., et al., The effects of FasL on inflammation and tumor survival are dependent on its expression levels. Cancer Gene Ther, 2007. 14(3): p. 262-7.
50. Kershaw, M. H., et al., A phase I study on adoptive immunotherapy using gene-modified T cells for ovarian cancer. Clin Cancer Res, 2006. 12(20 Pt 1): p. 6106-15.
51. Park, J. R., et al., Adoptive transfer of chimeric antigen receptor re-directed cytolytic T lymphocyte clones in patients with neuroblastoma. Mol Ther, 2007. 15(4): p. 825-33.
52. Louis, C. U., et al., Antitumor activity and long-term fate of chimeric antigen receptor-positive T cells in patients with neuroblastoma. Blood, 2011. 118(23): p. 6050-6.
53. Park, J. H. and R. J. Brentjens, Adoptive immunotherapy for B-cell malignancies with autologous chimeric antigen receptor modified tumor targeted T cells. Discov Med, 2010. 9(47): p. 277-88.
54. Kochenderfer, J. N., et al., Eradication of B-lineage cells and regression of lymphoma in a patient treated with autologous T cells genetically engineered to recognize CD19. Blood, 2010. 116(20): p. 4099-102.
55. Porter, D. L., et al., Chimeric antigen receptor-modified T cells in chronic lymphoid leukemia. N Engl J Med, 2011. 365(8): p. 725-33.
56. Kalos, M., et al., T cells with chimeric antigen receptors have potent antitumor effects and can establish memory in patients with advanced leukemia. Sci Transl Med, 2011. 3(95): p. 95ra73.
57. Brentjens, R. J., et al., Safety and persistence of adoptively transferred autologous CD19-targeted T cells in patients with relapsed or chemotherapy refractory B-cell leukemias. Blood, 2011. 118(18): p. 4817-28.
58. Kebriaei, P., et al., Infusing CD19-directed T cells to augment disease control in patients undergoing autologous hematopoietic stem-cell transplantation for advanced B-lymphoid malignancies. Hum Gene Ther, 2012. 23(5): p. 444-50.
59. Kochenderfer, J. N., et al., B-cell depletion and remissions of malignancy along with cytokine-associated toxicity in a clinical trial of anti-CD19 chimeric-antigen-receptor-transduced T cells. Blood, 2012. 119(12): p. 2709-20.
60. Till, B. G., et al., CD20-specific adoptive immunotherapy for lymphoma using a chimeric antigen receptor with both CD28 and 4-1BB domains: pilot clinical trial results. Blood, 2012. 119(17): p. 3940-50.
61. Brentjens, R. J., et al., CD19-targeted T cells rapidly induce molecular remissions in adults with chemotherapy-refractory acute lymphoblastic leukemia. Sci Transl Med, 2013. 5(177): p. 177ra38.
62. Kochenderfer, J. N. and S. A. Rosenberg, Treating B-cell cancer with T cells expressing anti-CD19 chimeric antigen receptors. Nat Rev Clin Oncol, 2013. 10(5): p. 267-76.
63. Davila, M. L. and R. Brentjens, Chimeric antigen receptor therapy for chronic lymphocytic leukemia: what are the challenges? Hematol Oncol Clin North Am, 2013. 27(2): p. 341-53.
64. Cruz, C. R., et al., Infusion of donor-derived CD19-redirected virus-specific T cells for B-cell malignancies relapsed after allogeneic stem cell transplant: a phase 1 study. Blood, 2013. 122(17): p. 2965-73.
65. Kochenderfer, J. N., et al., Donor-derived CD19-targeted T cells cause regression of malignancy persisting after allogeneic hematopoietic stem cell transplantation. Blood, 2013. 122(25): p. 4129-39.
66. Wu, R., et al., Adoptive T-cell therapy using autologous tumor-infiltrating lymphocytes for metastatic melanoma: current status and future outlook. Cancer J, 2012. 18(2): p. 160-75.
67. Kandalaft, L. E., D. J. Powell, Jr., and G. Coukos, A phase I clinical trial of adoptive transfer of folate receptor-alpha redirected autologous T cells for recurrent ovarian cancer. J Transl Med, 2012. 10: p. 157.
68. Lamers, C. H., et al., Treatment of metastatic renal cell carcinoma with CAIX CAR-engineered T cells: clinical evaluation and management of on-target toxicity. Mol Ther, 2013. 21(4): p. 904-12.
69. Schuberth, P. C., et al., Treatment of malignant pleural mesothelioma by fibroblast activation protein-specific re-directed T cells. J Transl Med, 2013. 11: p. 187.
70. van Schalkwyk, M. C., et al., Design of a phase I clinical trial to evaluate intratumoral delivery of ErbB-targeted chimeric antigen receptor T-cells in locally advanced or recurrent head and neck cancer. Hum Gene Ther Clin Dev, 2013. 24(3): p. 134-42.

1. A method of treating cancer comprising the steps of: a) obtaining a cell population from peripheral blood; b) transfecting said population with a chimeric antigen receptor (CAR); c) introducing said transfected cell population into said patient; and d) inhibiting NR2F6 activity to an extent sufficient to enhance T cell activation.

2. The method of claim 1, wherein said blood cell population is selected from a group comprising of: a) peripheral blood mononuclear cells; b) CD4 T cells; c) CD8 T cells; d) NK cells; e) NKT cells; and f) gamma delta T cells.

3. The method of claim 2, wherein said CD4 T cells are isolated by means of magnetic separation prior to transfection with CAR.

4. The method of claim 2, wherein said CD8 T cells are isolated by means of magnetic separation prior to transfection with CAR.

5. The method of claim 1, wherein said CAR is comprised of: a) an antigen binding domain; b) a transmembrane domain; c) a costimulatory signaling region; d) a CD3 zeta signaling domain.

6. The method of claim 5, wherein said CD3 zeta chain is resistant to cleavage by caspase 3 by means of amino acid substitution.

7. The method of claim 5, wherein the antigen binding domain is an antibody or an antigen-binding fragment thereof.

8. The method of claim 7, wherein the antigen-binding fragment is a Fab or a scFv.

9. The method of claim 5, wherein the antigen binding domain binds to a tumor specific and/or tumor associated antigen.

10. The method of claim 9, wherein said tumor specific and/or tumor associated antigen is selected from a group of antigens comprising of: a) HER2; b) CD19; c) EGFR; d) CD20; e) MUC1; and f) CD105.

11. The method of claim 5, wherein said costimulatory signaling region comprises the intracellular domain of a costimulatory molecule selected from the group comprising of CD27, CD28, 4-1BB, OX40, CD30, CD40, PD-1, ICOS, lymphocyte function-associated antigen-1 (LFA-1), CD2, CD7, LIGHT, NKG2C, B7-H3, a ligand that specifically binds with CD83.

12. The method of claim 1, wherein said transfected cell population is allogeneic to the cancer patient in need of treatment.

13. The method of claim 1, wherein said transfected cell population is autologous to the cancer patient in need of treatment.

14. The method of claim 1, wherein an inhibitor of a CD3 inhibitory molecule is co-administered together with the CAR.

15. The method of claim 14, wherein said inhibitor of CD3 inhibitory molecule is a dominant negative CTLA-4.

16. The method of claim 14, wherein said inhibitor of CD3 inhibitory molecule is a dominant negative IL-10 receptor.

17. The method of claim 14, wherein said inhibitor of CD3 inhibitory molecule is a dominant negative TGF-beta receptor.

18. The method of claim 1, wherein said CAR transfected cells are cotransfected with an a molecule capable of inducing RNA interference.

19. The method of claim 18, wherein said molecule capable of inducing RNA interference are selected from a group comprising of: a) siRNA or b) shRNA.

20. The method of claim 19, wherein silencing of molecules that inhibit CD3 zeta signaling are silenced.

21. The method of claim 20, wherein silencing of molecules is achieved, said molecules selected from a group comprising of: a) OX2; b) TGF-beta receptor; c) SMAD4; d) IL-10 receptor; e) PD-1; and f) CTLA-4.

22. The method of claim 18, wherein silencing of NR2F6 is achieved through introduction of introducing a short hairpin loop RNA (shRNA) comprising a sense sequence of 5′-GAT CCG CAT TAC GGT GTC TTC ACC TTC AAG AGA GGT GAA GAC ACC GTA ATG CTT TTT TCT AGA G-3′ or a sense sequence of 5′-GAT CCG CCT CTG GAC ACG TAA CCT ATT CAA GAG ATA GGT TAC GTG TCC AGA GGT TTT TTC TAG AG-3′
